(19) United States
(12) Patent Application Publication
(10) Pub. No.: US 2008/0214954 A1
(43) Pub. Date: Sep. 4, 2008

(54) Biopsy Device

(75) Inventor: Armando Belluomo, Ostia-Lido (Roma) (IT)

Correspondence Address: NIXON & VANDERHYE, PC, 901 NORTH GLEBE ROAD, 11TH FLOOR, ARLINGTON, VA, 22203, US

(73) Assignee: H.S. HOSPITAL SERVICE S.p.A., Roma (IT)

(21) Appl. No.: 11/883,762

(22) PCT Filed: Feb. 9, 2006
(86) PCT No.: PCT/EP06/50817

§ 371 Date: Aug. 6, 2007

(30) Foreign Application Priority Data
Feb. 11, 2005 (IT) MO2005 A 000033

(51) Int. Cl. A61B 10/02

(52) U.S. Cl. 600/567

(57) ABSTRACT

A biopsy device comprising a body, a mandrel provided with housing intended to receive a sample of organic tissue, a concentric hollow needle outside the mandrel, said mandrel and said hollow needle being slidingly coupled with said body and being movable in a direction parallel to the respective longitudinal axes between a respective proximal position and a respective distal position, a distal portion of said mandrel and of said hollow needle protruding outside said body; the biopsy device further comprises a concentric cannula outside said hollow needle, said cannula having sufficient length to cover completely said distal portion of said mandrel and said distal portion of hollow needle when the mandrel and the hollow needle are in a preset intermediate position between said distal position and said proximal position.

The present invention relates to a biopsy device of the guillotine type, i.e. to an instrument for taking samples of fragments of organs or tissues from patients, to diagnose the nature of a pathological process. The biopsy device according to the present invention is suitable, in particular, for taking samples of soft and/or compressible tissues.

From the state of the art, biopsy devices of the so-called “guillotine” type are known, that comprise a substantially cylindrical mandrel, near the tip of which a housing is obtained—for example obtained by making a flattening along a portion of the mandrel—intended to receive the sample to be taken, and a hollow needle, or cannula, with a cutting tip, slidingly coupled outside said mandrel. The housing has dimensions such as to receive a sample of tissue of sufficient size for the histological examinations to be conducted thereupon. The mandrel and the hollow needle are fixed to a movable body that controls the respective displacements of mandrel and hollow needle. The movable body is inserted inside a grip that enables the operator to operate the biopsy device.

In order to conduct the biopsy, the device is introduced into the patient's body, with the mandrel retracted inside the hollow needle so that only the tip emerges. When the tip of the mandrel has reached the zone of the patient's body from which the sample has to be taken, the mandrel is made to emerge from the hollow needle by mutual axial sliding. In this way, a portion of the tissue surrounding the mandrel surrounds the housing obtained on the mandrel. At this point, the hollow needle is advanced until it covers said housing so that the cutting point of the hollow needle separates from the surrounding tissue, with a guillotine action, the portion of tissue that has penetrated into the aforementioned housing. Lastly, the device is extracted from the patient's body.

If the biopsy has been conducted on an infected tissue, during retraction of the device from the patient's body there is the risk that the so-called “seeding” phenomenon will occur. The word “seeding” literally means insemination and refers to the possibility of spreading infected cells inside other healthy tissues in the retraction of the needle that has conducted the biopsy.

In fact, when the hollow needle is advanced to separate a portion of tissue from the surrounding tissue and trap it in the mandrel housing, the tip of the mandrel, which has been in contact with possible infected cells, remains outside the hollow needle.

During extraction of the biopsy device from the patient's body, a certain number of cells belonging to the zone in which the biopsy was conducted and which have remained adhering to the tip of the mandrel and to the distal end of the hollow needle, in numbers of varying amount according to various factors (roughness, correct cannula-mandrel coupling, etc) can spread in non-infected tissues that are between the zone in which the biopsy was conducted and the skin of the patient.

Biopsy devices, still of the guillotine type, are known that have particular shapes and geometries that, however, have steps, recesses, tips or localised section variations that increase enormously the risk that the phenomenon of “seeding” will occur and therefore the risk of causing even lethal infections for the patient.

A further drawback that occurs in the performance of a biopsy consists of the fact that, if the mandrel, or the hollow needle, during their advance in the patient's body, meet excessive resistance, the entire device may move back which may not be perceived by the operator. This moving back causes incorrect positioning of the device in the patient's body, with the consequence that the taking of the tissue sample may occur in a zone other than the preset one, which makes it necessary to repeat the biopsy. Furthermore, during the phase of advancing of the hollow needle it may be the mandrel and the device used as grips that move back, which causes failed cutting of the tissue or an only partial cutting thereof.

As in general it is unlikely that the mandrel will encounter excessive resistance during penetration, because the mandrel is contained almost completely inside the hollow needle and only the end part thereof emerges from the hollow needle during advancing of the mandrel, this risk is particularly high for the hollow needle. In fact, the hollow needle has all or a good part of the external surface thereof immersed in the tissues; the external surface is therefore subjected to a more or less high pressure field, depending on the type of tissue. At the moment in which the hollow needle starts advancing, a static friction is created that depends on the pressure exerted on the needle by the surrounding tissue, by the static friction coefficient between needle and tissue and by the extent of the surface of the needle inside the tissue. In addition to this friction, which is usually called stiction, the hollow needle encounters during advancing a dynamic friction that differs from static friction by the different value of the dynamic friction coefficient with respect to the static friction coefficient. Both the static friction and the dynamic friction are oppose the regular advance of the hollow needle and often produce the undesired effect of a moving back of the mandrel and of the body of the device. This effect is undoubtedly deleterious for the biopsy; in fact, as the hollow needle does not advance, there is no possibility of cutting the tissue and thus the biopsy is not successful and often has to be repeated.

The present invention aims to remedy the drawbacks specified above.

According to an aspect of the present invention a biopsy device is provided comprising a body inside which there is arranged a mandrel provided with housing intended to receive a sample of organic tissue and a hollow needle externally coaxial with the mandrel, said mandrel and said hollow needle being movable in a direction parallel to the respective longitudinal axes between a respective proximal position and a respective distal position, a distal portion of said mandrel and of said hollow needle protruding outside said body, characterised in that it furthermore comprises a cannula externally coaxial with said hollow needle, said cannula having a sufficient length to cover completely said distal portion of said mandrel and said distal portion of hollow needle, when the hollow needle is in said proximal position.

Owing to the presence of the cannula, when the hollow needle is advanced from the proximal position thereof to the distal position thereof, to separate a portion of tissue from the surrounding tissue and entrap it in the housing of the mandrel, only a short end portion of the hollow needle protrudes from the cannula and is subject to friction with the surrounding tissue, so that resistance to the advancing of the hollow needle is minimal and the risk of moving back of the device is substantially eliminated and, consequently, the risk of taking the bioptic sample in an incorrect position and also the risk of not taking a sufficient sample to conduct the subsequent analyses correctly are substantially eliminated.

In a further embodiment of the invention, the cannula is of sufficient length to cover completely both the distal portion of the hollow needle and the distal portion of the mandrel when they are both in the respective proximal positions.

This enables the mandrel and the hollow needle to be retracted completely inside the cannula, before extracting the device from the patient's body, so that possible infected cells that remain adhering to the mandrel and to the hollow needle remain inside the cannula whilst the device is extracted from the patient's body, thus making contamination of non-infected tissues impossible.

Another important advantage of the possibility of covering the distal ends of the hollow needle 8 and mandrel 6 is the elimination of the risk that the operator could hurt himself with the cutting surfaces of the mandrel or of the hollow needle that are presumed to be infected.

In a still further embodiment of the invention, the cannula is fixed in a removable manner to the body of the device. The possibility of separating the cannula from the body of the device makes it possible, after inserting the device into the patient's body and carrying out the biopsy, to leave the cannula in situ, so that it can act as a guide for carrying out further biopsies where they are necessary.

The invention will now be disclosed, purely by way of non-limitative example, with reference to the attached drawings, in which:

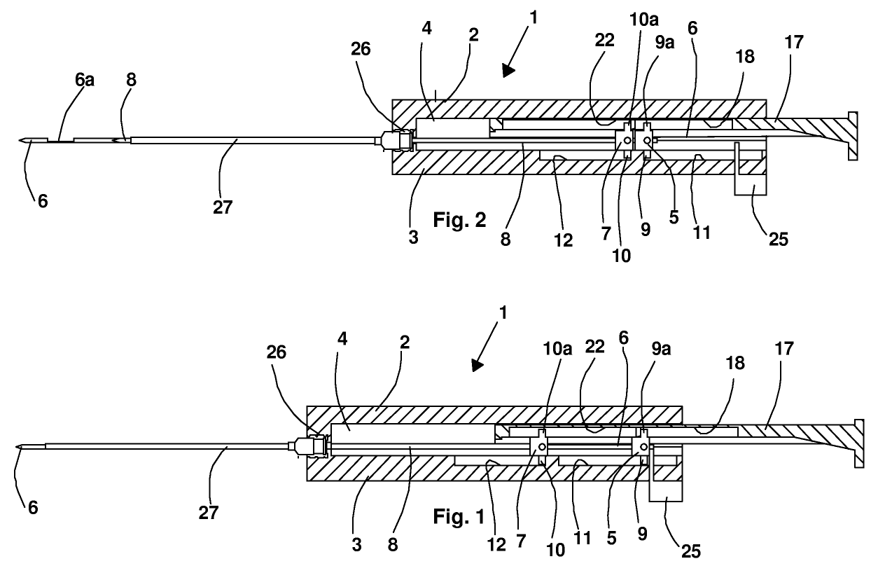

FIG. 1 is a longitudinal section of an embodiment of the device according to the invention, in a first operating position;

FIG. 2 is a longitudinal section of the device in FIG. 1, in a second operating position;

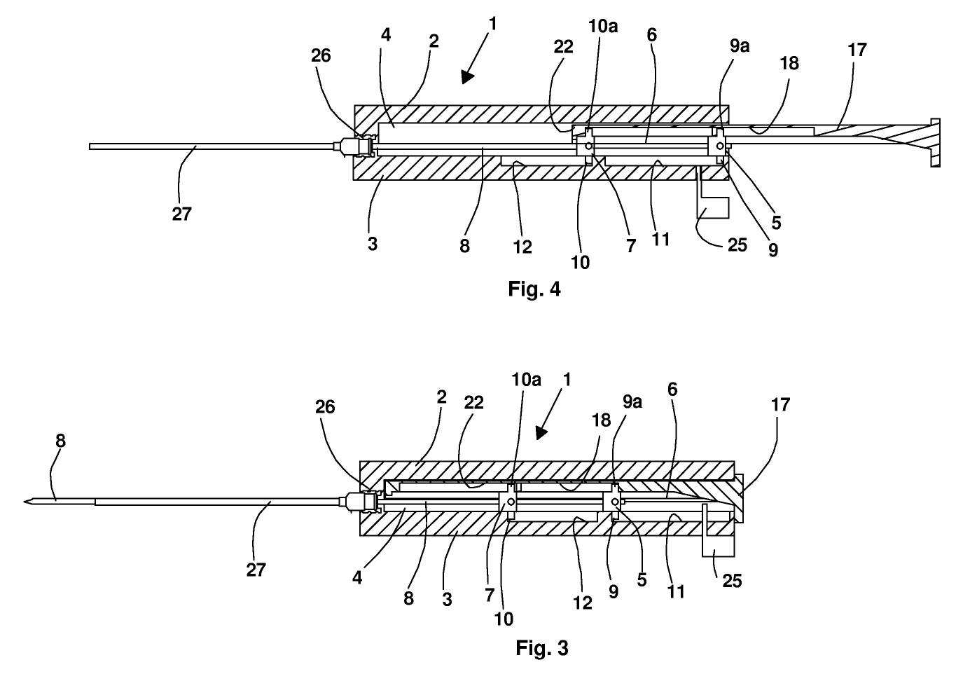

FIG. 3 is a longitudinal section of the device in FIG. 1, in a third operating position;

FIG. 4 is a longitudinal section of the device in FIG. 1, in a fourth operating position;

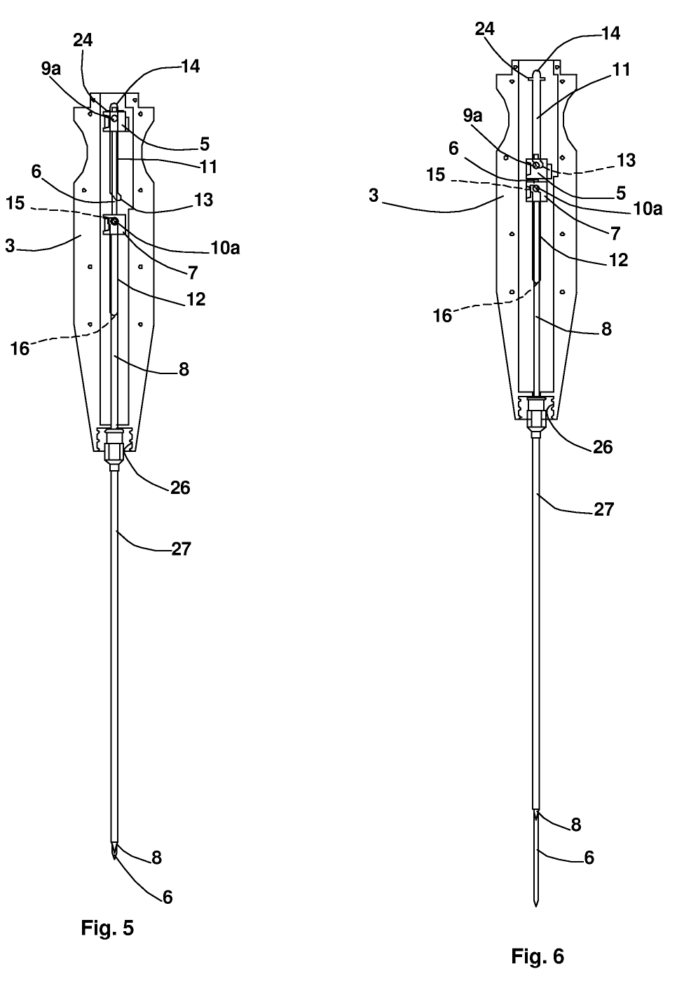

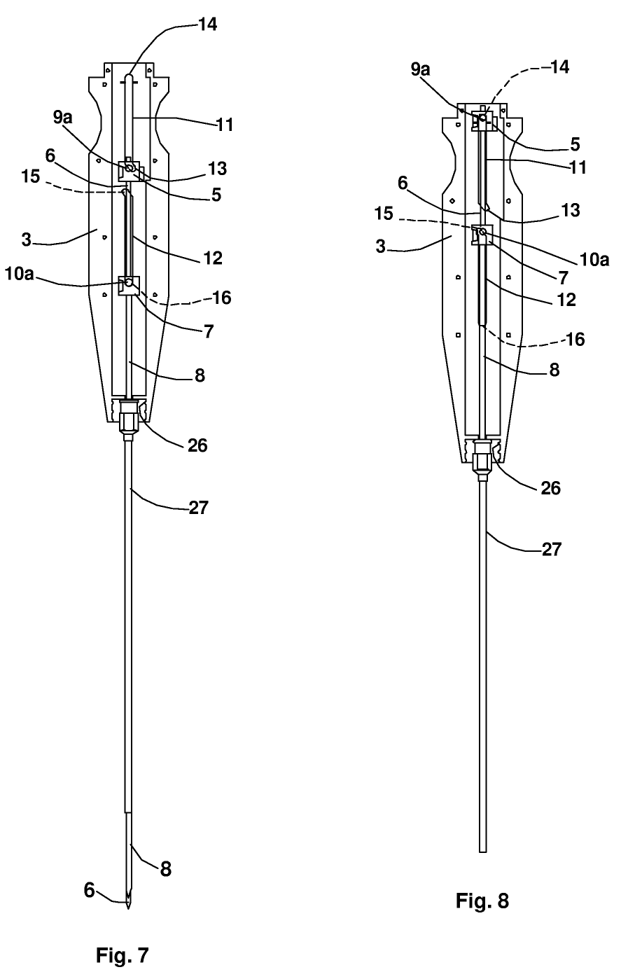

FIGS. 5 to 8 are views from above of the device in FIGS. 1 to 4, respectively;

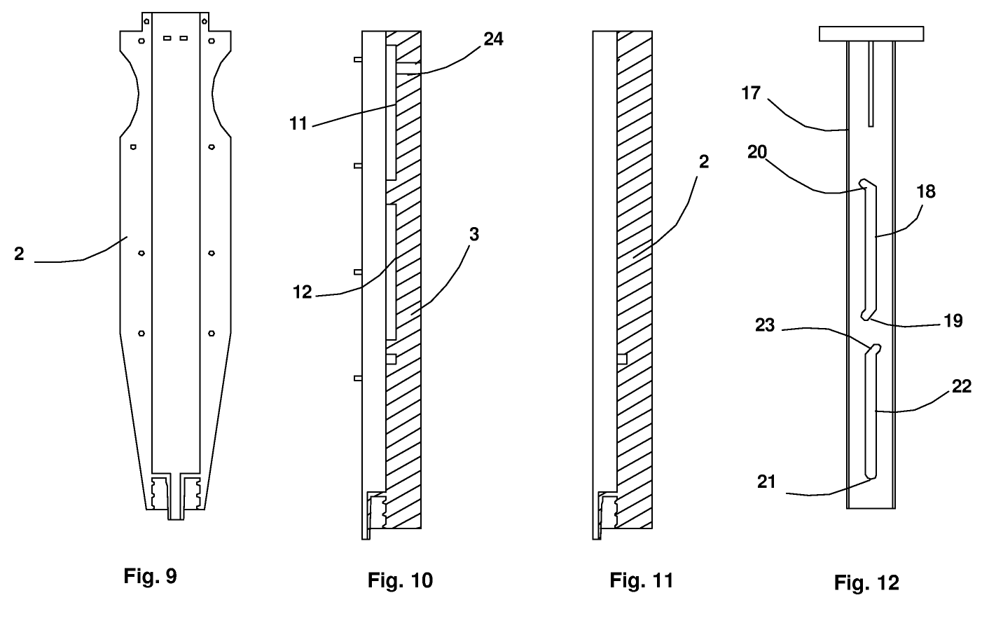

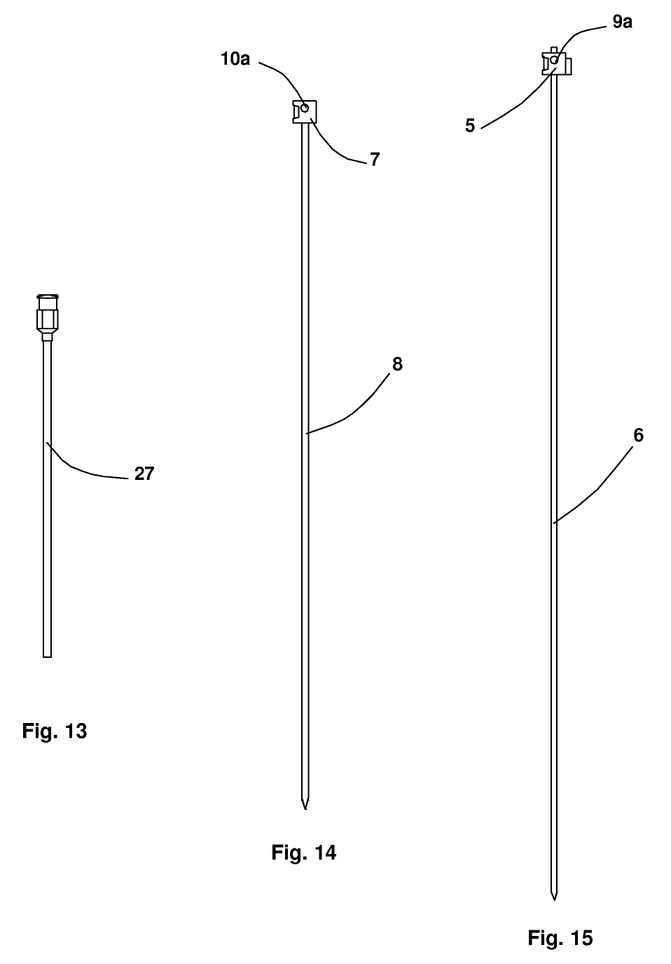

FIGS. 9 to 15 illustrate components of the device in FIGS. 1 to 4;

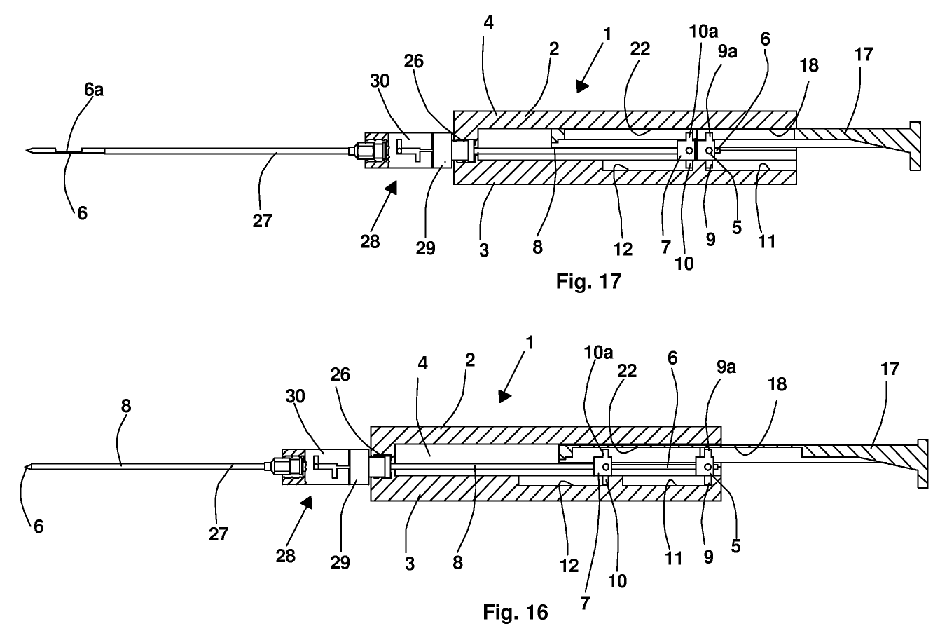

FIG. 16 is a longitudinal section of a further embodiment of the device according to the invention, in a first operating position;

FIG. 17 is a longitudinal section of the device in FIG. 16, in a second operating position;

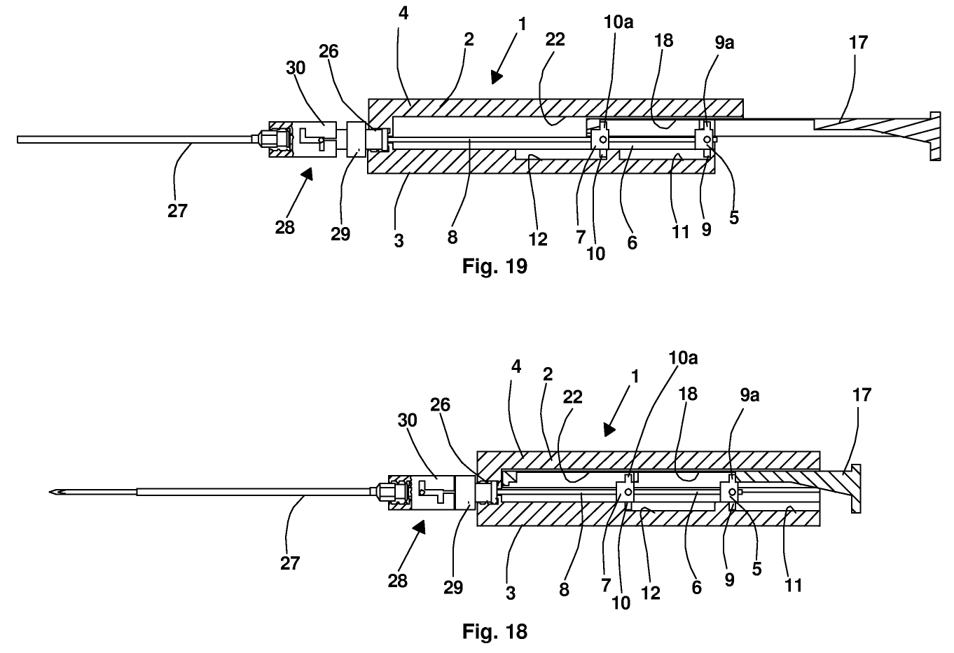

FIG. 18 is a longitudinal section of the device in FIG. 16, in a third operating position;

FIG. 19 is a longitudinal section of the device in FIG. 16, in a fourth operating position;

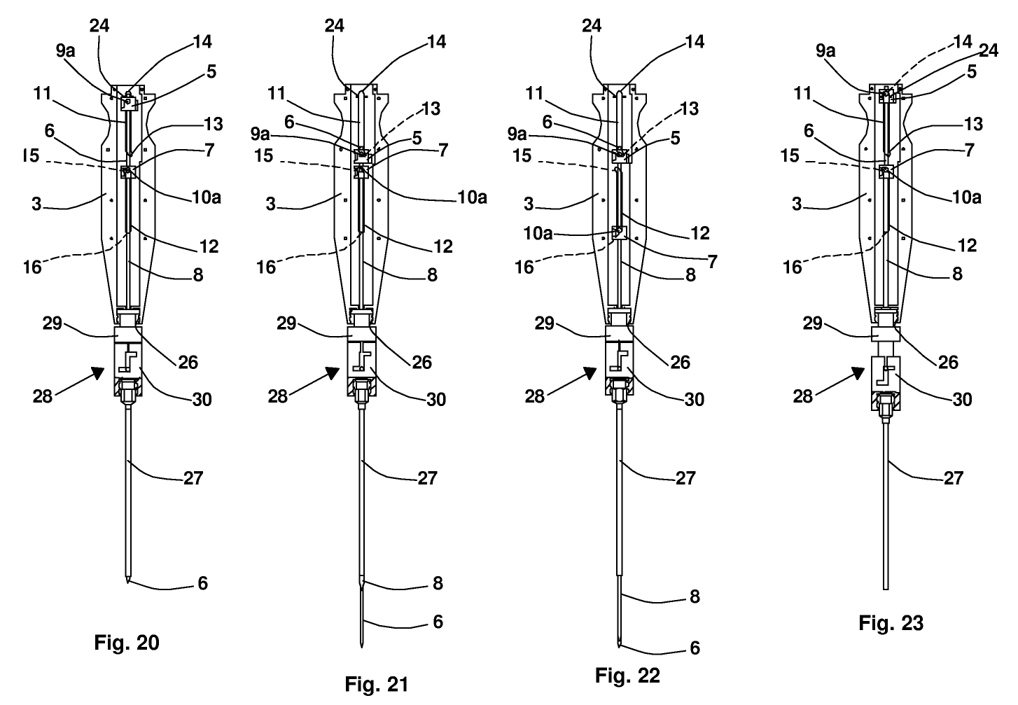

FIGS. 20 to 23 are views from above of the device in FIGS. 16 to 19, respectively;

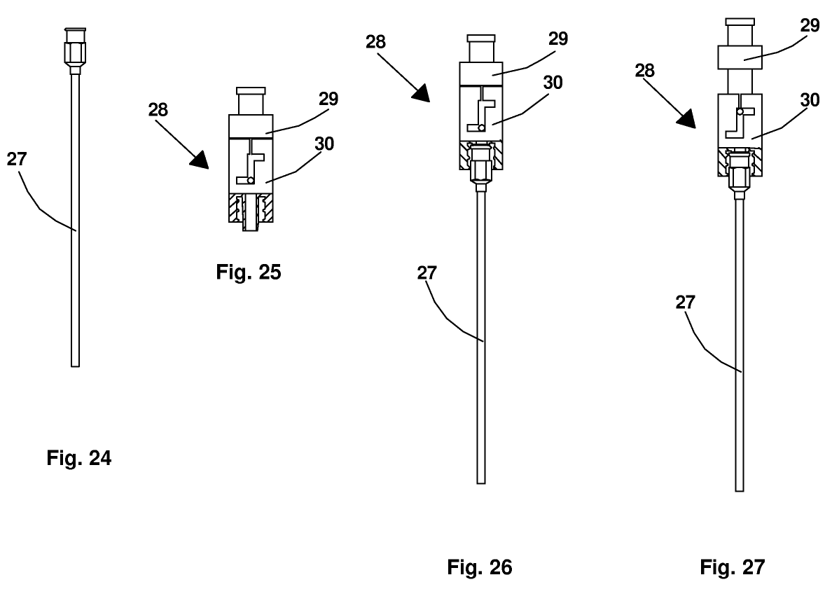

FIGS. 24 to 27 show details of the device in FIGS. 16 to 19.

With reference to FIGS. 1 to 15, a biopsy device according to the invention comprises a body 1 consisting of a first body part 2 and a second body part 3, which are coupled together. Inside the body 1, between said first part 2 and said second part 3 a cavity 4 is defined inside which a first movable element 5 is slidable to which there is fixed a proximal end of a mandrel 6, and a second movable element 7 to which a proximal end of a hollow needle 8 is fixed that surrounds the mandrel 6 externally.

The mandrel 6 is provided, near the proximal end thereof, with a seat 6a intended to receive a sample of organic tissue.

The first movable element 5 and the second movable element 7 are slidable in a direction substantially parallel to a longitudinal axis of the mandrel 6 and of the hollow needle 8.

The first movable element 5 is provided with a first appendage 9 and with a second appendage 9a, opposite said first appendage 9. The appendages 9, 9a extend in a direction substantially perpendicular to said longitudinal axis.

The second movable element 7 is also provided with a first appendage 10 and with a second appendage 10a, opposite said first appendage 10. The appendages 10, 10a extend in a direction substantially perpendicular to said longitudinal axis.

In the second body part 3 a first groove 11 and a second groove 12 are obtained that are both communicating with said cavity 4 and aligned in a direction substantially parallel to said longitudinal axis. The first groove 11 is intended to be coupled with the first appendage 9 of the first movable element 5 during the movements of the latter inside the cavity 4. The first groove 11 terminates, at a distal end thereof, with a notch 13 (FIG. 5), offset laterally with respect to said longitudinal axis, intended to receive and lock said first appendage 9, in a distal position of the first movable element 5, that defines a distal position of the mandrel 6. An end 14 of the groove 11 opposite said notch 13 defines a proximal stop position for the first movable element 5 that defines a proximal position of the mandrel 6. The second groove 12 is intended to couple with the first appendage 10 of the second movable element 7 during the movement of the latter inside the cavity 4. The second groove 12 terminates, at the proximal end thereof, with a notch 15, offset laterally with respect to said longitudinal axis, intended to receive and lock said first appendage 10, in a proximal stop position of the second movable element 7, that defines a proximal position of the hollow needle 8. An end 16 of the groove 12, opposite said portion 15, defines a distal stop position for the second movable element 7 that defines a distal position of the hollow needle 8.

The first movable element 5 and the second movable element 7 are drivable to slide inside the cavity 4 by a driving element 17 that is slidingly couplable, as a piston, inside the cavity 4.

The driving element 17 is provided with a first groove 18 that extends in a direction substantially parallel to said longitudinal axis and is intended to be coupled with the second appendage 9a of the first movable element 5. The first groove 18 terminates, at a distal end thereof, with a first notch 19, offset laterally with respect to said longitudinal axis, intended to guide said first movable element 5 to the distal stop position previously mentioned. The first groove 18 furthermore terminates at a proximal end thereof with a second notch 20, offset laterally with respect to said longitudinal axis, intended to guide said first movable element 5 to the proximal stop position mentioned previously.

The driving element 17 is furthermore provided with a second groove 22 substantially aligned with said first groove 18 in said direction parallel to said longitudinal axis. The second groove 22 is intended to be coupled with the second appendage 10a of the second movable element 7 and terminates at a proximal end thereof with a notch 23 offset laterally with respect to said longitudinal axis, intended to guide said second movable element 7 to the distal stop position previously mentioned.

In the second part 3 of the body 1 a through hole 24 is made, orientated in a direction substantially perpendicular to said longitudinal axis. In the opening 24 a stop element 25 is slidable that defines an intermediate stop position for the first movable element 5, between said proximal stop position and said distal stop position. The distance of the intermediate stop position from the proximal stop position of the movable element 5 is preferably less than the distance of the intermediate stop position from the distal stop position of the movable element 5.

At said distal end of the body 1 a seat 26 is obtained in which it can be fixed in a removable manner, for example with a coupling of the so-called “luer-lock” type, a proximal end of a cannula 27 that externally surrounds the mandrel 6 and the hollow needle 8, being coaxial with them (the coupling between the seat 26 and a proximal end of a cannula 27 can also be envisaged fixed and obtained through a simple luer cone glued to the surfaces of the seat). The length of the cannula 27 is chosen so that it is able to cover completely a distal portion of hollow needle 8 that protrudes from the body 1, when the hollow needle 8 is in the proximal position thereof.

In order to conduct a biopsy, the device 1 is initially in a first operating position, illustrated in FIG. 1, in which the first movable element 5 is in the intermediate stop position against the stop element 25 and the second movable element 7 is in the proximal stop position, with the first appendage 10 engaged and locked in the notch 15 of the second groove 12 of the second element 3 of the body 1. The driving element 17 is positioned so that the second appendage 9a of the first movable element 5 is engaged and locked in the first notch 19 of the first groove 18.

In this first operating position, the distal portion of hollow needle 8 that protrudes from the body 1 is completely inside the cannula 27 from which only the tip of the mandrel 6 protrudes.

With the device in this first operating position, the mandrel 6, the hollow needle 8 and the cannula 27 are introduced into the body of a patient until the distal end of the mandrel 6 reaches the zone in which the biopsy has to be conducted.

When the distal end of the mandrel 6 has reached the zone in which the biopsy has to be conducted, the driving element 17 is advanced to the distal end of the body 1, which causes an advance to said distal end of the first movable element 5, inasmuch as the second appendage 9a thereof, locked in the first notch 19 of the first groove 18 of the driving element 17, is dragged by the latter, thus causing the advance of the first movable element 5. Advancing of the driving element continues until the first appendage 9 of the first movable element 5 reaches the distal end of the first groove 11 of the lower part 3 of the body 2, engaging and locking in the notch 13.

Advancing the first movable element 5 causes advancing of the mandrel 6 that emerges from the cannula 27 until it completely uncovers the seat 6a, so as to enable a portion of organic tissue to surround said seat 6a (FIG. 2).

At this point, the driving element 17 is advanced further to the distal end of the body 1 of the device. This further advancing causes the disengagement of the second appendage 9a of the first movable element 5 from the first notch 19 of the first groove 18, whilst the second appendage 10a of the second movable element 7 engages and remains locked in the notch 23 of the second groove 22 of the driving element 17. In this way, the further advance of the driving element 17 determines a corresponding advance of the second movable element 7, whilst the first movable element 5 remains stationary in the distal stop position.

Advancing of the driving element 17 and of the second movable element 7 terminates when the first appendage 10 of the second movable element 7 reaches the distal end of the second groove 12 of the second part 3 of the body 1.

The advancing of the second movable element 7 causes corresponding advancing of the hollow needle 8, the distal end of which emerges from the cannula 27 and advances until it cover completely the seat 6a of the mandrel 6 (FIG. 3). A portion of tissue that surrounds the seat 6a is cut by the distal end of the hollow needle 8 and remains trapped in the seat 6a, inside the hollow needle 8.

Before extracting the device according to the invention from the patient's body, the stop element 25 moves towards the outside of the body 2, so that it no longer interferes with the first movable element 5. The driving element 17 is then moved away from the distal end of the body 2 until the second movable element 7 is brought to its proximal stop position and the first movable element 5 is brought to its proximal stop position, as shown in FIG. 4. This causes simultaneous movement of the mandrel 6 and of the hollow needle 8 towards the proximal end of the body 2 until they reach the respective proximal positions, in which the distal ends of the mandrel 6 and of the hollow needle 8 are both completely inside the cannula 27 (FIG. 4).

In this condition, the device can be extracted from the patient's body without the risk that possible infected cells coming from the zone of the biopsy and remaining adhering to the distal end of the mandrel 6 and/of the hollow needle 8 may contaminate healthy tissues that are located along the extraction trajectory of the device from the patient's body. This is because the cannula 27 completely shields the distal ends of the mandrel 6 and of the hollow needle 8, preventing said possible infected cells being able to be deposited on healthy tissues.

The cannula 27 furthermore protects the operator from the risk of coming into contact with cutting surfaces presumed to be infected.

During this phase, in the event of removable coupling, it is possible to decouple the cannula 27 from the device, leaving it in situ in the patient's body to act as a guide if further bioptic samples need to be taken from the same zone of the patient's body.

In FIGS. 16 to 27 there is illustrated another embodiment of a device according to the invention, in which covering of the distal ends of the mandrel 6 and of the hollow needle 8 is achieved by making the cannula 27 movable in a direction parallel to the longitudinal axis of the mandrel and of the hollow needle.

In this embodiment of the device the stop element 25 is not present, so no intermediate stop position of the movable element 5 is defined between the distal stop position thereof and the proximal stop position thereof. The cannula 27 is coupled in a removable manner, through, for example, a coupling of the “luer-lock” type to an operating element 28 fixed to the seat 26 of the body 2, the latter also, for example, with coupling of the “luer-lock” type (also in this case the above couplings can be made fixed, for example through gluing). The operating element 28 consists of a first part 29, fixed to said seat 26 and of a second part 30 slidingly coupled with said first part, so as to be able to slide in relation to it in a direction substantially parallel to said longitudinal axis, so as to vary the length of the operating element 28 and consequently, the position of the cannula 27 in relation to the mandrel 6 and to the hollow needle 8.

The operation of this embodiment is illustrated in FIGS. 16 to 23.

In order to carry out a biopsy, the device 16 starts in a first operating position, illustrated in FIG. 1, in which both the first movable element and the second movable element 7 are in the respective proximal stop positions with the first appendage 10 of the second movable element 7 engaged and locked in the notch 15 of the second groove 12 of the second element 3 of the body 1. The driving element 17 is positioned so that the second appendage 9a of the first movable element 5 is engaged and locked in the first notch 19 of the first groove 18.

In this first operating position, the distal portion of hollow needle 8 that protrudes from the body 1 is completely inside the cannula 27, from which only the tip of the mandrel 6 protrudes. In this first operating position it is also possible to provide for the end part of the distal portion of the hollow needle, at the cutting surfaces of the latter element, protruding outside the surfaces of the cannula 27.

With the device in this first operating position, the mandrel 6, the hollow needle 8 and the cannula 27 are inserted into the body of a patient until the distal end of the mandrel 6 reaches the zone in which the biopsy has to be conducted.

When the distal end of the mandrel 6 has reached the zone in which the biopsy has to be conducted, the driving element 17 is advanced towards the distal end of the body 1, which causes an advance towards said distal end of the first movable element 5, inasmuch as the second appendage 9a thereof, locked in the first notch 19 of the first groove 18 of the driving element 17, is dragged by the latter, thus causing the advance of the first movable element 5. The advance of the driving element continues until the first appendage 9 of the first movable element 5 reaches the distal end of the first groove 11 of the lower part 3 of the body 2, engaging and locking in the notch 13.

Advancing of the first movable element 5 causes an advance of the mandrel 6 that emerges from the cannula 27 until it uncovers the seat 6a completely, so as to enable a portion of organic tissue to penetrate into said seat 6a (FIG. 17).

At this point, the driving element 17 is advanced further towards the distal end of the body 1 of the device. This further advance causes the disengagement of the second appendage 9a of the first movable element 5 from the first notch 19 of the first groove 18, whilst the second appendage 10a of the second movable element 7 engages and remains locked in the notch 23 of the second groove 22 of the driving element 17. In this way, the further advance of the driving element 17 causes a corresponding advance of the second movable element 7, whilst the first movable element 5 remains stationary in the distal stop position.

The advancing of the driving element 17 and of the second movable element 7 terminates when the first appendage 10 of the second movable element 7 reaches the distal end of the second groove 12 of the second part 3 of the body 1.

The advance of the second movable element 7 causes a corresponding advance of the hollow needle 8, the distal end of which emerges from the cannula 27 and advances until it cover completely s the seat 6a of the mandrel 6 (FIG. 18). A portion of tissue that surrounds the seat 6a is cut by the distal end of the hollow needle 8 and remains entrapped in the seat 6a, inside the hollow needle 8.

Before extracting the device according to the invention from the patient's body, it is necessary to release the operating device 28 so that the two elements constituting it, i.e. the element 30 integral with the cannula 27 and the element 29 integral with the body 1, have the possibility of moving in relation to one another. After the release, it is necessary to make the body 1 move back, keeping the element 30 stationary until the element 29 reach the distal stop obtained on the element 30. With this operation it is possible to insert the mandrel 6 and the hollow needle 8 (FIG. 19) completely inside the cannula 27.

In this condition the device can be extracted from the patient's body without the risk that possible infected cells coming from the zone of the biopsy and remaining adhering to the near end of the mandrel 6 and/or of the hollow needle 8 may contaminate the healthy tissues that are located along the extraction trajectory of the device from the patient's body.

In the practical embodiment, the materials, dimensions and constructional details may be different from those indicated but be technically equivalent thereto without thereby falling outside the scope of the present invention, as defined by the claims.

1-24. (canceled)

25. A biopsy device for taking a sample of organic tissue comprising a body, a mandrel provided with a housing intended to receive a sample of organic tissue, a hollow needle externally coaxial with the mandrel and a cannula externally coaxial with said hollow needle, said mandrel and said hollow needle being designed to be inserted into said organic tissue, said mandrel and said hollow needle being slidingly coupled with said body and being movable in a direction parallel to the respective longitudinal axes between a respective proximal position and a respective distal position, a distal portion of said mandrel and a distal portion of said hollow needle protruding outside said body, wherein said cannula is designed to be inserted into said organic tissue together with said mandrel and said hollow needle during the taking of said sample, said cannula having a sufficient length to cover at least a substantial part of the distal portion of said hollow needle when the hollow needle is in said proximal position.

26. The device according to claim 25, wherein said cannula has a sufficient length to cover completely the distal portion of said hollow needle and the distal portion of said mandrel, when both the hollow needle and the mandrel are in the respective proximal positions.

27. The device according to claim 25, wherein said cannula is removably coupled with said body.

28. The device according to claim 25, wherein said cannula is irremovably coupled with said body.

29. The device according to claim 25, wherein said body comprises a first body part and a second body part between which there is defined a cavity.

30. The device according to claim 29, wherein a proximal end of said mandrel is fixed to a first movable element suitable for sliding in said cavity in a direction substantially parallel to longitudinal axes of said mandrel and said hollow needle.

31. The device according to claim 30, wherein said first movable element is provided with a first appendage and with a second appendage both extending in a direction substantially perpendicular to said longitudinal axes.

32. The device according to claims 29, wherein a proximal end of said hollow needle is fixed to a second movable element suitable for sliding in said cavity in a direction substantially parallel to said longitudinal axes.

33. The device according to claim 32, wherein said second movable element is provided with a first appendage and with a second appendage both extending in a direction substantially perpendicular to said longitudinal axes.

34. The device according to claim 31, wherein said second part of said body is provided with a first groove suitable for being coupled with the first appendage of the first movable element.

35. The device according to claim 34, wherein said second part of said body is provided with a second groove suitable for being coupled with the first appendage of the second movable element, said second groove being aligned on said first groove in a direction substantially parallel to said longitudinal axes.

36. The device according to claim 35, wherein said first groove is provided, at a distal end thereof, with a notch offset laterally with respect to said direction.

37. The device according to claim 35, wherein said second groove is provided, at a proximal end thereof, with a notch offset laterally with respect to said direction.

38. The device according to claim 32, further comprising a driving element that is slidable inside said cavity in a direction substantially parallel to said longitudinal axes.

39. The device according to claim 38, wherein said driving element comprises a first groove orientated in a direction substantially parallel to said longitudinal axes and suitable for being coupled with the second appendage of said first movable element.

40. The device according to claim 39, wherein said driving element comprises a second groove aligned with said first groove and suitable for being coupled with the second appendage of said second movable element.

41. The device according to claim 39, wherein said first groove is provided, at a distal end thereof with a first notch offset laterally with respect to said direction.

42. The device according to claim 39, wherein said first groove is provided at a proximal end thereof with a second notch offset laterally with respect to said direction.

43. The device according to claim 40, wherein said second groove is provided, at a proximal end thereof, with a notch offset laterally with respect to said direction.

44. The device according to claim 31, wherein with said second part of said body is associated with a stop element suitable for interacting with said first movable element.

45. The device according to claim 44, wherein said stop element is slidable in an opening of said second part, in a direction substantially perpendicular to said longitudinal axes.

46. The device according to claim 25, wherein said cannula is coupled with said body by means of an operating element of variable length.

47. The device according to claim 46, wherein said operating element comprises a first part coupled with a seat provided in said body and a second part slidingly coupled with said first part, so as to be able to slide with respect to it in a direction substantially parallel to said longitudinal axes between a proximal position and a distal position, said cannula being coupled with said second part.

48. The device according to claim 47, wherein said cannula has sufficient length to cover completely said hollow needle and said mandrel when both the hollow needle and the mandrel are in the respective proximal positions and said second part of the operating element is in the respective distal position.
